(19) United States
(12) Patent Application Publication
(10) Pub. No.: US 2015/0064290 A1
(43) Pub. Date: Mar. 5, 2015

(54) CULTIVATED MOMORDICA SPECIES AND EXTRACT THEREOF

(71) Applicant: Nutritional Health Institute Laboratories, LLC, Tallahassee, FL (US)

(72) Inventor: Konky Sotomayor, Palm Harbor, FL (US)

(73) Assignee: NUTRITIONAL HEALTH INSTITUTE LABORATORIES, LLC, Tallahassee, FL (US)

(21) Appl. No.: 14/472,162

(22) Filed: Aug. 28, 2014

Related U.S. Application Data
(60) Provisional application No. 61/871,171, filed on Aug. 28, 2013.

(51) Int. Cl. A61K 36/42; A23L 1/222; A01H 5/08

(52) U.S. Cl. CPC A61K 36/42; A01H 5/08; A23L 1/222
USPC 424/758; 800/309

(57) ABSTRACT

A plant grown from seeds derived from a new interhybrid of a hybrid of three plants of the Momordica genus and a second hybrid of two plants of the Momordica genus and a plant of the Siraitia genus has been developed. Parts of these new hybrid plants, when ingested by a human, have blood glucose and/or insulin regulating properties as well as anti-inflammatory properties. Edibles containing parts of the plants or extracts that promote blood glucose regulation and/or act as an anti-inflammatory are also disclosed.

This application claims priority to U.S. patent application Ser. No. 61/871,171, filed Aug. 28, 2013, the contents of which are hereby incorporated by reference as if fully set forth herein.

### FIELD OF THE INVENTION

The present invention relates to a new plant hybrid of the genus Momordica, and, more particularly, to a new Momordica interhybrid, parts of which can be extracted to produce a material having improved blood sugar and/or insulin regulatory properties in humans.

### BACKGROUND

Blood glucose levels play an important role in a person's energy levels and, in general, all bodily functions. A continuous excess of glucose in a person's blood stream is often an indicator of weight and/or blood glucose regulation issues. Generally, an individual's blood glucose levels should fall between about 70 to about 110 mg/dl.

Elevated blood glucose levels (i.e., greater than about 110 mg/dl) typically triggered by alimentary habits, can have a severe impact on an individual's health and can lead to a wide range of problems. For example, diabetes, heart disease, stroke, high blood pressure and blindness are some of the complications that can be derived from prolonged high blood glucose levels. Diabetes, in particular, has been shown to be a precursor of kidney and nervous system disorders and can also cause dental disease and complications of pregnancy.

Recent research has indicated that obesity is associated with a state of chronic, low-grade inflammation, particularly in white adipose tissue. In recent years, much has been learned about the intracellular signaling pathways activated by inflammatory and stress responses and how these pathways intersect with and inhibit insulin signaling.

Insulin affects cells through binding to its receptor on the surface of insulin-responsive cells. The stimulated insulin receptor phosphorylates itself and several substrates, including members of the insulin receptor substrate (IRS) family, thus initiating downstream signaling events. The inhibition of signaling downstream of the insulin receptor is a primary mechanism through which inflammatory signaling leads to insulin resistance. Recently it has become clear that inflammatory signaling pathways can also become activated by metabolic stresses originating from inside the cell as well as by extracellular signaling molecules.

It has been demonstrated that obesity overloads the functional capacity of the endoplasmic reticulum (ER) and that this ER stress leads to the activation of inflammatory signaling pathways and thus contributes to insulin resistance. Additionally, increased glucose metabolism can lead to a rise in mitochondrial production of reactive oxygen species (ROS). ROS production is elevated in obesity, which causes enhanced activation of inflammatory pathways.

Blood sugar imbalances attributable to diabetes are typically treated or controlled using insulin or insulin mimetics. For patients who are pre-diabetic and/or are non-insulin dependent, other forms of treatment or intervention may be employed to control blood glucose levels. For example, lifestyle changes that include weight management, nutritional control and/or counseling, and/or exercise regimens may be prescribed or recommended.

As noted, insulin and insulin mimetics are often used to treat or control the symptoms associated with diabetes. Anti-inflammatory agents also may be used in connection with or as adjuvant therapies to control or alleviate the symptoms associated with diabetes-related disease processes such as, for example, diabetic retinopathy, diabetic neuropathy, and/or diabetic nephropathy amongst others. However, some of the insulin mimetics and anti-inflammatory agents used to manage diabetic disease processes have low bioavailability in the human body due to lack of solubility in the gastro-intestinal tract.

In view of the above, there is a need and a desire for a product, such as a natural product or a new plant hybrid, that is effective for lowering blood glucose levels. Further, there is a need and a desire for an over-the-counter (OTC) product that can be utilized alone or in combination with other rehabilitative therapies to control blood glucose levels. There is an additional need and a desire for a natural product or new plant hybrid that can be used to supplement diet and exercise and/or promotes weight loss or control. There is a still further need and a desire for a natural product or new plant hybrid which is effective for controlling inflammatory processes which contribute to the development of insulin resistance. There is also a need and desire for a product isolated from a single source such as a new plant hybrid which provides both insulin regulating and inflammatory process regulating properties. There is yet another need and a desire for a natural product or new plant hybrid which improves and/or enhances the bioavailability of insulin regulating and/or anti-inflammatory agents.

### SUMMARY

A general object of the invention is to provide a naturally-derived product or product derived from a new plant hybrid which exhibits blood sugar and/or insulin regulation properties. A further general objective is to provide such a product which exhibits enhanced anti-inflammatory properties. A still further general objective is to provide such a product isolated from a single source which exhibits blood sugar and/or insulin regulation properties in combination with anti-inflammatory properties. Another general object is to provide a natural product, new plant hybrid or product derived from the new hybrid that has enhanced bioavailability and/or improves the bioavailability of compounds or compositions used in connection with the product. Yet another object is to provide a sweetener product which exhibits blood sugar and/or insulin regulating properties which may also exhibit enhanced anti-inflammatory properties.

The general object of the invention can be obtained, at least in part, through a new plant interhybrid of the genus Momordica an extract derived there from. The new interhybrid is made by cross-pollinating two hybrids, said hybrids being a first hybrid of Siraitia grosvenorii and Momordica cymbalaria and a second hybrid of Siraitia grosvenorii with Momordica dioca, and then cross-pollinating the resultant hybrid with a previously-developed Momordica hybrid to form a new interhybrid species. The new Momordica interhybrid extract is believed to control or regulate blood sugar and/or insulin levels within the body. The new Momordica interhybrid extract is further believed to have an anti-inflammatory effect on the body.

The invention further comprehends an edible product containing the extract derived from the new plant hybrid that assists in the regulation of blood glucose and/or insulin levels, mediation of inflammatory processes and/or can be utilized to promote weight loss or weight control.

Other objects and advantages will be apparent to those skilled in the art from the following detailed description taken in conjunction with the examples.

### DETAILED DESCRIPTION

The invention provides a newly derived plant hybrid of the genus Momordica. Extracts obtained or isolated from the new Momordica sp. possess blood sugar and/or insulin regulation properties alone or in combination with anti-inflammatory properties.

Terpene glycosides have been found in many plant species. Many of these compounds are physiologically active and possess a broad range of medico-biological action. For example, several terpene glycosides were discovered to enhance the solubility of a number of pharmaceutically and medicinally important compounds. These terpene glycosides are a naturally occurring class of water solubility-enhancing compounds that are non-toxic and may be useful as complexing agents or excipients. Without being bound by this theory, it is believed that the improved solubility of water-insoluble drugs is a result of the formation of terpene glycoside-drug complex structures, such as nano- to micro-size micelles, which are water soluble.

Natural terpene glycosides generally are terpene aglycones attached to at least one glucose, and the most common forms are diterpene glycosides and triterpene glycosides. Many of these compounds are also known to be non-toxic and natural sweeteners.

Extracts obtained from certain species of the Momordica genus have been shown to have a regulatory impact upon blood glucose and/or insulin levels in human subjects. In some cases it is believed that such extracts can successfully restore Beta cells' ability to secrete insulin. In the present invention, a new interhybrid of the genus Momordica has been cultivated to have an improved growth rate thereby providing an economical and readily accessible supply of plant material.

Extracts obtained from the Siraitia grosvenorii plant (also known as Luo Han Guo) have been found to exhibit anti-inflammatory properties. In particular, mogrosides, triterpene glycosides derived from the fruit of the plant, may inhibit inflammation induced by lipopolysaccharides by down-regulating the expression of key inflammatory genes iNOS, COX-2, and IL-6 and up-regulating some inflammation protective genes such as PARP1, BCL211, TRP53, and MAPK9. Similarly, 12-O-tetradecanoylphorbol-13-acetate-induced inflammation may be inhibited by mogrosides by down-regulating COX-2 and IL-6 and up-regulating PARP1, BCL211, TRP53, MAPK9, and PPARδ gene expression.

In addition to anti-inflammatory properties, mogrosides, triterpene glycosides that make up approximately 1% of the flesh of the fresh fruit, and in particular mogroside V, are known to have sweetening properties and have been approved for use as a food grade sweetener.

This application is directed to a new double interhybrid of the plant described and claimed in U.S. Pat. No. 8,071,844 (hereinafter the “McB interhybrid” which was deposited with the American Type Culture Collection and assigned ATCC® Ascension No. PTA-11843), the contents of which are hereby incorporated by reference as if fully set forth herein, and the inter-hybrid created with other species of the same genus Momordica, cymbalaria and dioca, with the recently adopted name Siraitia grosvenorii, formerly known as Momordica grosvenorii.

In other words, the McB interhybrid is the interhybrid of Momordica charantia, Momordica balsamina and a Momordica species native from Ecuador. The McB interhybrid will be cross-pollinated with another inter-hybrid which parents are Momordica cymbalaria, Momordica dioca and Siraitia grosvenorii. For the purposes of coding, the second interhybrid will be hereinafter called “MSIH” for the first letters of Momordica-Siraitia interhybrid.

Following an already established protocol, MSIH will be derived from two double cross-pollinations of the second set of three parent species. Siraitia grosvenorii is bred with Momordica cymbalaria to create msih1. Siraitia grosvenorii is also bred with Momordica dioca to create msih2. Then msih1 is crossed with msih2 to create MSIH. Thereafter, the McB interhybrid is crossed with MSIH to derive the new inventive plant hybrid, which has yet to be named.

Instructions for Cross-Pollination of Momordica and Siraitia Flowers

1) Determine which flowers are male and which are female. The plants of the genus Momordica (or Momordica/Siraitia) in this application have male and female flowers on the same plant. Male flowers have stamen and anthers. Female flowers will have the pistil, and for flowers that turn into fruit, there will be a small ovary under the base of the flower that looks like a thickening of the stem.

In this first step we swab the anthers of the male flowers with a cotton swab or paint brush to collect the pollen. We then transfer the collected pollen to the stigma of a female flower in the next row.

Repeat this action until you have pollinated each flower.

2) The plants to be double cross-pollinated are set in separate rows for the first two species (i.e., a row of Siraitia grosvenorii and a row of Momordica cymbalaria) and are manually pollinated from the male flowers of one plant in row one to the female flowers of the other plant in row two and then manually pollinate from the male flowers of the plant in row two to the female flowers of the other plant in row one. This will produce a stage one hybrid msih1.

3) The process is duplicated using Siraitia grosvenorii and Momordica dioca and we derive a stage 2 hybrid msih2.

4) The process is again duplicated using msih1 and msih2 to derive interhybrid MSIH.

5) The final stage performed in order to derive what we would call a super inter-hybrid McB/MSIH is done by repeating the cross-pollination process already described using these later two.

Thereafter the plants grown from seeds derived from this new super interhybrid of five plants of the Momordica genus and one plant of the Siraitia genus can be obtained under this procedure.

There are then parts or extracts of the new interhybrid MSIH as well as parts of the super interhybrid plant which, when ingested by an individual, would have blood glucose and/or insulin regulating properties. As used herein, parts of a plant or parts of the plant include any portion of the plant, including (but not limited to) leaves, flowers, flower petals, stems, roots, fruits, seeds, and portions and/or combinations thereof. Foods and/or beverages, or other edibles such as a dietary supplement, containing parts or extracts of the new interhybrid plant MSIH and/or parts or extracts of the super interhybrid could be assumed to promote weight control or loss based on the disclosed blood glucose regulation capabilities.

It is believed that one or more metabolic pathway including the repair of Beta cell is present in the super interhybrid and that anti-inflammatory property would be exhibited by MSIH and the super interhybrid due to hybrid vigor introduced by the inclusion of Siraitia grosvenorii as a parent herb. This anti-inflammatory property is not found in currently known Momordica species or the previously patented Momordica hybrid. As inflammation can be a comorbid condition with blood sugar or insulin regulation problems, the new hybrids disclosed herein are believed to have the advantage of treating both conditions simultaneously. Thus there is also provided a method of treating blood glucose or insulin regulation disorders, inflammation, or both, by providing a patient in need thereof with a therapeutically effective amount of an extract of MSIH or the super interhybrid, parts of one of said hybrids, or an edible containing such extract or parts.

An extract of the MSIH interhybrid or the super interhybrid may be obtained via extraction of the plant material in a ratio of about 1 gram plant material to about 10 grams of extraction liquid. Typically, such extraction liquid may consist of or include, water, ethanol or a combination thereof. One suitable technique for obtaining an extract includes an aqueous soxhlet extraction method.

In accordance with one aspect, extracts from the above described interhybrids may be used to prepare a sweetener having blood glucose and/or insulin regulating properties alone or in combination with anti-inflammatory properties.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention. The above description incorporates preferred embodiments and examples as a means of describing and enabling the invention to be practiced by one of skill in the art. It is imagined that changes can be made without departing from the spirit and scope of the invention described herein and defined in the appended claims.

The MSIH interhybrid seeds, producing the newly derived plant hybrid and used in the methods disclosed herein are deposited with a depository such as the American Type Culture Collection (ATCC®), Patent Depository. It is expected that upon testing that the seeds are viable.

Likewise, the super inter-hybrid McB/MSIH seeds, producing a newly developed plant hybrid and used in the methods disclosed herein are deposited with a depository such as the American Type Culture Collection (ATCC®), Patent Depository. It is expected that upon testing that the seeds are viable.

What is claimed is:

1. A plant grown from a species interhybrid seed having an ATCC accession number derived from the cross-pollination of a first hybrid and second hybrid, with the first hybrid created from breeding Siraitia grosvenorii with Momordica cymbalaria, and the second hybrid created from breeding Siraitia grosvenorii with Momordica dioca.

2. A plant grown from a species interhybrid seed having an ATCC accession number derived from the cross-pollination of the plant claimed in claim 1 with a Momordica species interhybrid having ATCC accession number PTA-11843 derived from a tetra-cross pollination of parent species Momordica charantia, Momordica balsamina and a native Ecuadoran Momordica species.

3. Parts of a plant grown from a seed having an ATCC accession number derived from the cross-pollination of a first hybrid and second hybrid, with the first hybrid created from breeding Siraitia grosvenorii with Momordica cymbalaria, and the second hybrid created from breeding Siraitia grosvenorii with Momordica dioca.

4. Parts of a plant produced by a seed having an ATCC accession number derived from the cross-pollination of the plant claimed in claim 1 with a Momordica species interhybrid having ATCC accession number PTA-11843 derived from a tetra-cross pollination of parent species Momordica charantia, Momordica balsamina and a native Ecuadoran Momordica species.

5. An extract of a plant or parts of a plant grown from a species interhybrid seed having an ATCC accession number derived from the cross-pollination of a first hybrid and second hybrid, with the first hybrid created from breeding Siraitia grosvenorii with Momordica cymbalaria, and the second hybrid created from breeding Siraitia grosvenorii with Momordica dioca, the extract having blood glucose regulating properties, insulin regulating properties or a combination thereof.

6. An extract of a plant or parts of a plant grown from a species interhybrid seed having an ATCC accession number derived from the cross-pollination of the plant claimed in claim 1 with a Momordica species interhybrid having ATCC accession number PTA-11843 derived from a tetra-cross pollination of parent species Momordica charantia, Momordica balsamina and a native Ecuadoran Momordica species, the extract having blood glucose regulating properties, insulin regulating properties or a combination thereof.

7. A method of treating blood glucose or insulin conditions in a human subject, comprising:
providing to a patient in need thereof a therapeutically effective amount of the extract of claim 6.

8. The method of claim 7, wherein the extract is combined with an edible component.

9. The method of claim 8, wherein the extract combined with the edible component is a dietary supplement ingested by an individual to maintain blood glucose levels at a normal level or to reduce blood glucose levels.

10. A method of treating inflammation in a human subject, comprising:
providing to a patient in need thereof a therapeutically effective amount of the extract of claim 6.

11. The method of claim 10, wherein the extract is combined with an edible component.

12. The method of claim 11, wherein the extract combined with the edible component is a dietary supplement ingested by an individual to reduce inflammation.

13. The method of claim 7, wherein the extract is provided in the form of a sweetener.

14. The method of claim 10, wherein the extract is provided in the form of a sweetener.
